(19) United States
(12) Patent Application Publication
(10) Pub. No.: US 2010/0286090 A1
(43) Pub. Date: Nov. 11, 2010

(54) Treatment of Multiple Myeloma

(75) Inventors: Constantine S Mitsiades, Boston, MA (US); Qingwei Deng, Watertown, MA (US); Nicholas S. Mitsiades, West Roxbury, MA (US); Kenneth C. Anderson, Wellesley, MA (US); Joseph Negri, E. Falmouth, MA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: BRISTOL-MYERS SQUIBB COMPANY

(21) Appl. No.: 12/084,657

(22) PCT Filed: Nov. 14, 2006
(86) PCT No.: PCT/US06/44084

§ 371 Date: Jul. 20, 2010

Related U.S. Application Data
(60) Provisional application No. 60/736,390, filed on Nov. 14, 2005.
(60) Provisional application No. 60/748,522, filed on Dec. 8, 2005.

(51) Int. Cl. A61K 31/69; A61K 31/573; A61K 31/506; A61P 35/00

(52) U.S. Cl. 514/64; 514/171; 514/252.19

(57) ABSTRACT

Methods for treating multiple myeloma comprising administering a therapeutically effective amount of dasatinib to a patient in need of treatment thereof. Dasatinib can be administered alone or in combination with a second anti-neoplastic agent such as dexamethasone or bortezomib. The patient may be refractory to prior treatment with an anti-neoplastic agent other than dasatinib.

### FIELD OF THE INVENTION

The present invention relates generally to the field of the treatment of cancer, and more specifically to methods for treatment of multiple myeloma.

### BACKGROUND OF THE INVENTION:

Despite recent advances in the development of new classes of anti-cancer drugs (e.g., proteasome inhibitors, thalidomide, and thalidomide derivatives) for the treatment of multiple myeloma (MM), no curative therapy currently exists for this disease, which is the 2^{nd} most commonly diagnosed hematologic malignancy in the Western World. Therefore, the identification of new therapeutic agents with anti-MM activity remains an urgent priority.

The genetic heterogeneity of multiple myeloma (MM) and the evolution of the disease as it progresses result in a multiplicity of proliferative/anti-apoptotic pathways that can operate in MM cells, particularly within the context of their interaction with the bone marrow (BM) microenvironment. Collectively, these factors can contribute to de novo or acquired refractoriness of MM cells to diverse conventional and/or novel anti-MM therapeutics. To counteract the multiplicity of pathways potentially implicated in the control of MM cell resistance to drug-induced apoptosis, the use of multi-targeted small-molecule inhibitors (e.g., kinase inhibitors) has been explored, clinical levels of which can simultaneously suppress the expression of multiple targets.

### SUMMARY OF THE INVENTION

The invention provides a method for treating multiple myeloma comprising administering a therapeutically effective amount of dasatinib to a patient in need of treatment thereof.

In one aspect, the method comprises combination therapy of dasatinib and at least one other anti-neoplastic agent. In one aspect, the at least one other anti-neoplastic agent is selected from dexamethasone, alkylating agents, anthracyclines, thalidomide, immunomodulatory thalidomide derivatives, Apo2L/TRAIL, proteasome inhibitors, and cytotoxic chemotherapy anti-MM agents. In another aspect, the at least one other anti-neoplastic agent is dexamethasone or bortezomib.

In another aspect, the patient receiving dasatinib treatment is resistant to a prior multiple myeloma treatment.

In yet another aspect, the method comprises combination therapy of dasatinib and an HMG-CoA reductase inhibitor. In one aspect, the HMG-CoA reductase inhibitor is lovastatin.

The invention will be better understood upon a reading of the detailed description of the invention when considered in connection with the accompanying figures.

### BRIEF DESCRIPTION OF THE DRAWINGS

FIG. 1 illustrates results obtained on the effect of dasatinib at varying concentrations on the cell viability of certain MM cell lines.

FIG. 2 illustrates results which indicate that dasatinib overcomes the protective effect of stromal cells on MM cells.

FIG. 3 illustrates results which indicate that dasatinib induces caspase-8 activation in MM-1S cells.

FIG. 4 illustrates results obtained correlating the transcriptional profiles of MM cells with their degree of responsiveness to low nM levels of dasatinib.

### DETAILED DESCRIPTION OF THE INVENTION

Described herein are studies on the oral, multi-targeted kinase inhibitor dasatinib (BMS-354825, Bristol-Myers Squibb Co.) which inhibits BCR-ABL, SRC, c-KIT, PDGF-R, and ephrin (EPH) receptor kinases. Although BCR-ABL and c-KIT are not primary oncogenes driving MM proliferation and survival, dasatinib was studied because of (a) emerging data from our laboratory (CS Mitsiades, unpublished observations) on the expression patterns of EPH receptors in MM cell lines and primary tumor specimens; and (b) the roles of PDGF-R and SRC in tumor-microenvironment interactions, e.g., pericyte function in angiogenesis and osteoclast-mediated bone resorption, respectively. In vitro, it was found that dasatinib significantly suppresses, at clinically achievable sub-μM concentrations, the viability of MM cell lines (including lines resistant to conventional or other novel anti-MM agents), primary tumor specimens from multi-drug resistant MM patients, as well as MM cells co-cultured with BM stromal cells. Mechanistic studies showed that dasatinib-induced caspase-8 activation and sensitized primary MM cells to agents activating caspase-9 (e.g., dexamethasone (Dex) and bortezomib). Even though IC₅₀ values were higher in MM cells than in BCR-ABL⁺ CML cells, the dasatinib IC₅₀ was <100 nM in 8/15 MM cell lines tested, suggesting substantial sensitivity to dasatinib in at least a subset of MM cases. Interim analyses correlating the transcriptional profiles of MM cells with their degree of responsiveness to low nM levels of dasatinib showed that increased responsiveness to this inhibitor correlated with increased expression of diverse proliferative/anti-apoptotic genes, including transcriptional. regulators (e.g., MAF, MAFF, NFYC, PML, YY1, DAXX), cell surface receptors (e.g., EPH receptor B4, CXCR4), proteasome subunits (PSMC3, PSMD12, PSME2) and regulators of apoptosis (e.g., CIAP1, IKK-e).

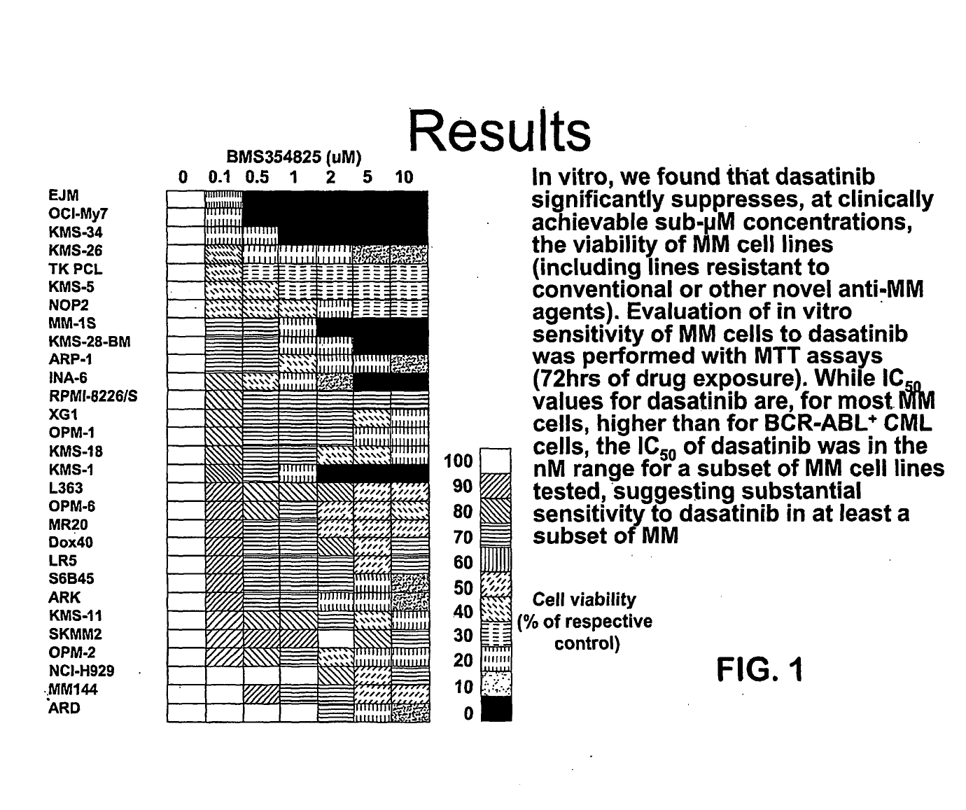

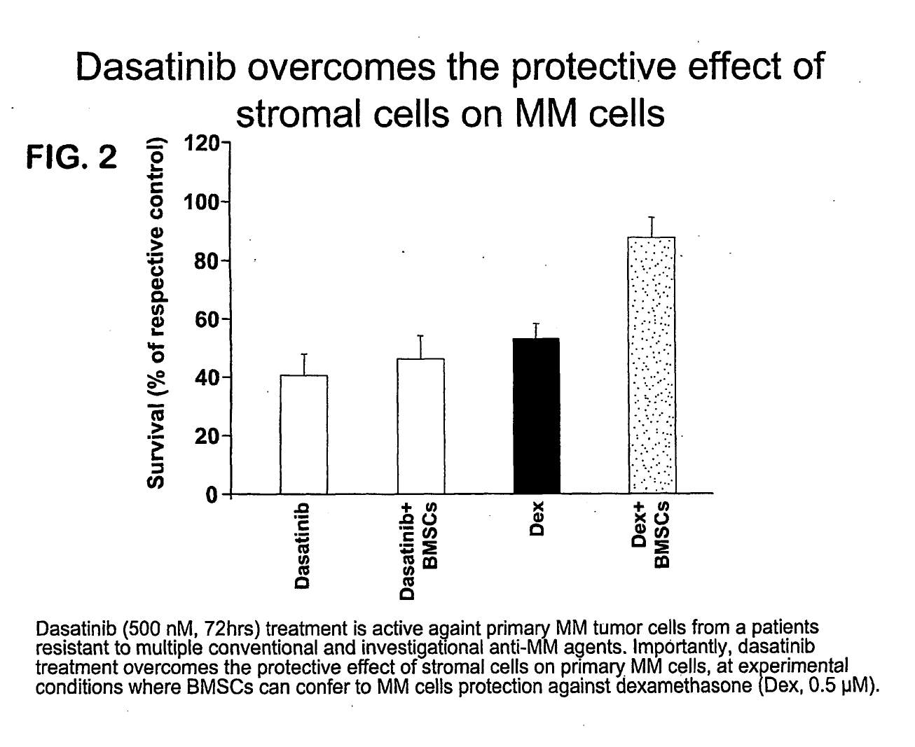

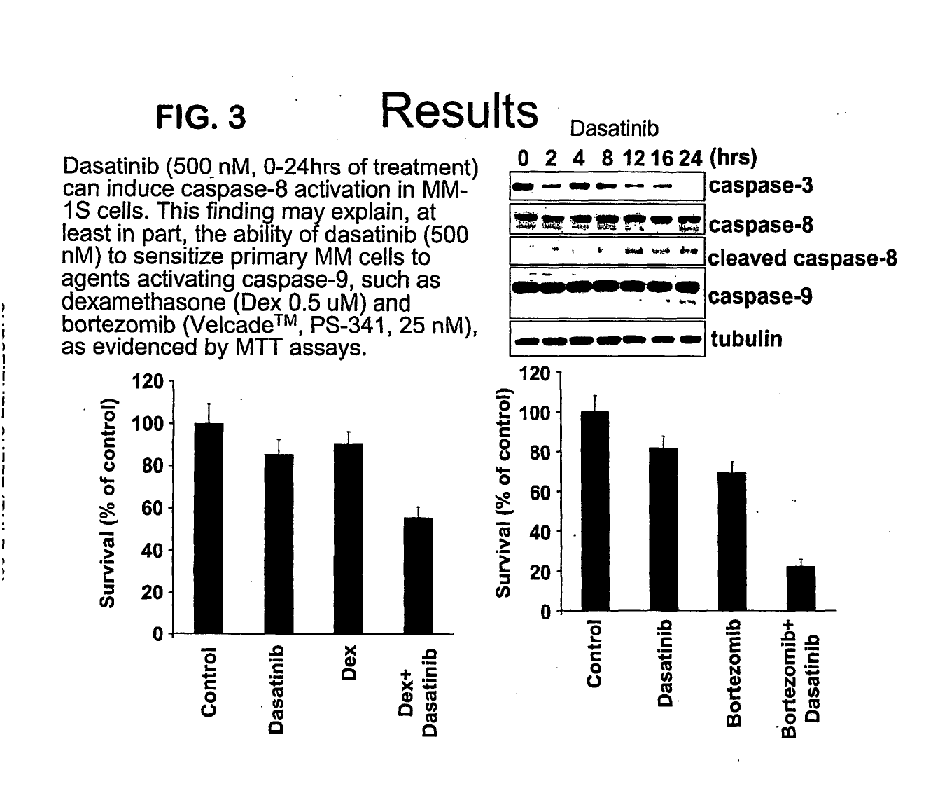

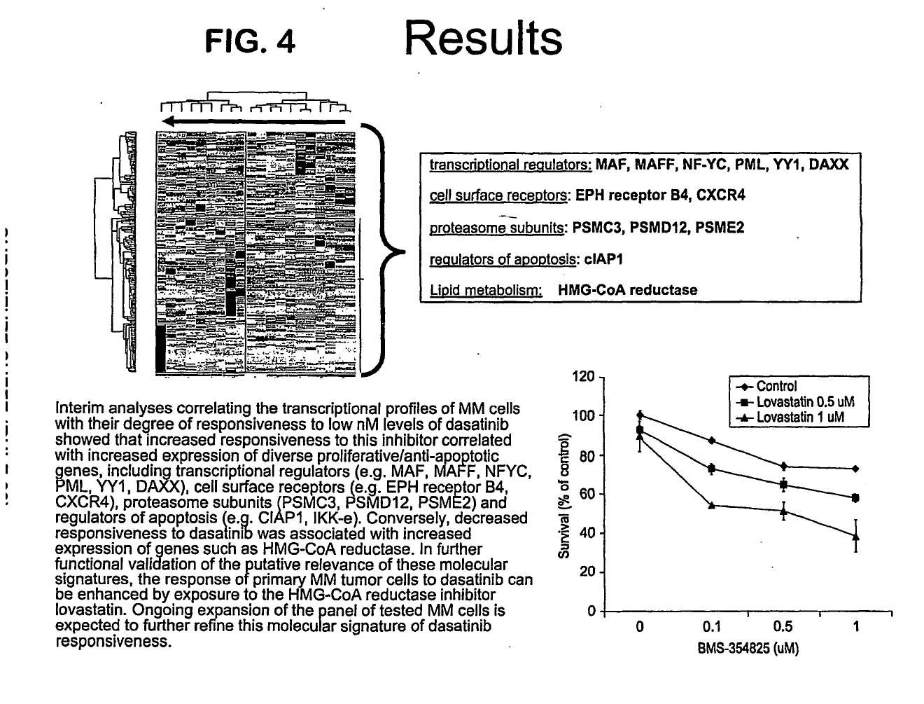

FIGS. 1-4 illustrate results of the invention. The results of these studies are as follows.

Dasatinib is active against human MM cells which are resistant to conventional or other investigational treatments. It was found that dasatinib has potent in vitro activity against a broad panel of human MM cell lines, which include MM cells sensitive or resistant to conventional (e.g., dexamethasone, alkylating agents, anthracyclines) or novel (e.g., thalidomide, immunomodulatory thalidomide derivatives, Apo2L/TRAIL) anti-MM agents. For those cell lines highly responsive to dasatinib, their IC₅₀ was in a range of concentrations which are deemed clinically achievable levels (based on data derived from the ongoing clinical trials of this compound in other disease setting). These results suggest that dasatinib can be active against a broad spectrum of different molecular subgroups of multiple myeloma patients.

Dasatinib is active against drug-resistant primary MM tumor cells. The experiments show that the in vitro anti-MM activity of dasatinib is not restricted only to cell lines, but is also documented against primary MM tumor cells isolated from patients resistant to conventional therapies (e.g., dexamethasone, cytotoxic chemotherapy) or more recently introduced therapies for MM (e.g., thalidomide or its analogs and/or proteasome inhibition), further supporting the finding that dasatinib can be an active agent for the treatment of a broad spectrum of MM patients, including those with de novo or acquired resistance to currently used conventional or investigational therapies.

Dasatinib overcomes the protective effect of bone marrow stromal cells (BMSCs) on MM cells. The anti-MM activity of conventional anti-cancer drugs (e.g., steroids, cytotoxic chemotherapy) is attenuated when MM cells interact with BMSCs. However, it was found that in the setting of co-culture of MM cells with BMSCs, the treatment with dasatinib is able to overcome the protective effect of the BMSCs, indicating that treatment of MM with dasatinib can be active in cases were tumor cells develop resistance to conventional drugs because of tumor-stromal interactions.

Dasatinib sensitizes MM cells to other anti-myeloma agents. It was found that in vitro dasatinib treatment enhances the response of primary MM cells to other anti-myeloma agents, including cytotoxic chemotherapeutics or proteasome inhibitors, indicating that dasatinib treatment can be combined with other investigational agents or with conventional anti-myeloma therapeutics.

What is claimed is:

1. A method for treating multiple myeloma comprising administering a therapeutically effective amount of dasatinib to a patient in need of treatment thereof.

2. The method of claim 1, wherein said treating further comprises administration of an anti-neoplastic agent.

3. The method of claim 2 wherein said anti-neoplastic agent is dexamethasone.

4. The method of claim 2 wherein said anti-neoplastic agent is bortezomib.

5. The method of claim 1 wherein said patient is resistant to treatment of multiple myeloma with at least one anti-neoplastic agent.

6. The method of claim 1 wherein said treating further comprises administration of an HMG-CoA reductase inhibitor.

7. The method of claim 1 wherein said HMG-CoA reductase inhibitor is lovastatin.
